(19) United States
(12) Patent Application Publication
(10) Pub. No.: US 2005/0065557 A1
(43) Pub. Date: Mar. 24, 2005

(54) Method and apparatus for printing incident review data from an external defibrillator without the need of a computer

(76) Inventors: Daniel J. Powers, Issaquah, WA (US); Catherine Ann Thompson, Seattle, WA (US)

Correspondence Address: ATL ULTRASOUND, P.O. BOX 3003, 22100 BOTHELL EVERETT HIGHWAY, BOTHELL, WA, 98041-3003, US

(21) Appl. No.: 10/895,182

(22) Filed: Jul. 20, 2004

Related U.S. Application Data
(60) Provisional application No. 60/504,407, filed on Sep. 19, 2003.

(51) Int. Cl. A61N 1/39

(52) U.S. Cl. 607/5; 600/518

(57) ABSTRACT

A method and apparatus is provided for reviewing incident data on an external defibrillator (10). The method begins by deploying the defibrillator (10) for use in an emergency, wherein the defibrillator (10) is attached to a patient (18). The ECG data from the patient (18) is monitored and then recorded in an image file located in a memory device (22) incorporated in the defibrillator (10).

This invention claims the benefit of Provisional U.S. Patent Application Ser. No. 60/504,407, filed Sep. 19, 2003.

The present invention relates in general to defibrillators, and more particularly to automatic or semi-automatic external defibrillators (“AED”).

Sudden cardiac death is the leading cause of death in the United States, with one person dying every two minutes. Most sudden cardiac death is caused by ventricular fibrillation (“VF”), in which the heart's muscle fibers contract without coordination, thereby interrupting normal blood flow to the body. The only known effective treatment for VF is electrical defibrillation, in which an electrical pulse is applied to the patient's heart. The electrical pulse must be delivered within a short time after onset of VF in order for the patient to have any reasonable chance of survival. Electrical defibrillation may also be used to treat shockable ventricular tachycardia (“VT”). Accordingly, defibrillation is the appropriate therapy for any shockable rhythm, i.e., VF or shockable VT.

One way of providing electrical defibrillation uses an external defibrillator. External defibrillators send electrical pulses to the patient's heart through electrodes applied to the patient's torso. External defibrillators are typically located and used in hospital emergency rooms, operating rooms, and emergency medical vehicles. Of the wide variety of external defibrillators currently available, automatic and semi-automatic external defibrillators (referred to collectively as “AEDs”) are becoming increasingly popular because they can be used by relatively inexperienced personnel. Such AEDs are also especially lightweight, compact, and portable. AEDs are described in U.S. Pat. No. 5,607,454 to Cameron et al. entitled “Electrotherapy Method and Apparatus,” PCT Publication No. WO 94/27674 entitled “Defibrillator with Self-Test Features,” and U.S. Published Patent Application No. 2002/0156503 entitled “Method and Apparatus for Providing On-Screen Incident Review in an AED,” the specifications of which are incorporated herein.

AEDs provide a number of advantages, including the availability of external defibrillation at locations where external defibrillation is not regularly expected, and is likely to be performed quite infrequently, such as in residences, public buildings, businesses, personal vehicles, public transportation vehicles, etc. Because AEDs are designed to be small, lightweight and easy to maintain, AEDs generally do not feature a paper based ECG recorder.

One drawback to using an AED is that it typically does not provide a way to review the historical ECG data since AEDs display only the currently monitored information. ECG review is accomplished via a paper-based ECG print-out. Because of the added weight associated with an ECG printer this is a feature that is typically only included in the larger multi-feature defibrillators (for example, the CodeMaster 100 by Hewlett-Packard). As AEDs have become more common (e.g. in airports, hotels, cruise ships, and airplanes), the number of times responsibility for the care of a victim is transferred increases. For example, instead of the traditional paramedic to emergency room transfer, the situation now exists where a first responder (such as a lay person, or airline attendant) may be superceded by a firefighter or police officer and then a paramedic prior to being taken to the emergency room at a hospital. Each time responsibility for a patient is handed off to a more advanced caregiver, it is important to be able to quickly transfer relevant historical treatment information. However, while professional caregivers are accustomed to providing a thumbnail summary of prior treatment to an advanced caregiver, such as an emergency room physician, the same is not true for a lay responder.

Thus, when emergency response personnel are called to the scene of a cardiac arrest or a patient is transferred to the emergency room, the ability to quickly review the incident (or ECG history) is desirable since the initial lay responder may not be in a position to accurately describe the early portions of the treatment. This is particularly important, because ECG history can impact subsequent treatment. Further, it is desirable to note the various treatments applied at the scene and record the patient's response. Such information might help trained cardiologists, reviewing the information, to rule out certain disease or defect conditions that potentially could afflict the patient prior to administering treatment.

Instead of a built-in printer, some AEDs store the ECG history on a data card that must be inserted into the card reader of a computer. The computer then sends the data to a printer that is connected to the computer by a parallel or USB cable, or by a network wired or wireless connection. In any case, a computer is necessary to review the ECG history. One drawback to printing the ECG history in this manner is that many of the places where an AED would be located such as fire stations, airplanes and the like do not have a computer readily available.

Accordingly, it would be desirable to provide an AED in which ECG data can be printed without the need for either a paper-based ECG recorder or a computer. In accordance with the present invention, a method and apparatus is provided for reviewing incident data on an external defibrillator. The method begins by deploying the defibrillator for use in an emergency, wherein the defibrillator is attached to a patient. The ECG data from the patient is monitored and the recorded in an image file located in a memory device incorporated in the defibrillator.

In accordance with one aspect of the invention, the memory device is user removable, and the recorded ECG data is printed by removing the memory device from the defibrillator and inserting the memory into a memory reader incorporated in a printer.

In accordance with another aspect of the invention, the recording step includes recording audible data received from a microphone.

In accordance with another aspect of the invention, the recording step includes recording defibrillator operation information.

In accordance with another aspect of the invention, the external defibrillator is a fully automatic external defibrillator.

In accordance with another aspect of the invention, the external defibrillator is a semi-automatic external defibrillator.

In accordance with another aspect of the invention, the ECG image file is transmitted from the defibrillator to a display device.

In accordance with another aspect of the invention, additional data is selected to be recorded in the image file located on the memory.

In accordance with another aspect of the invention, the additional data is selected from the group consisting of audible data and patient therapy data.

In accordance with another aspect of the invention, the memory device is selected from the group consisting of a compact FLASH, an ATA flash memory card, a Memory Stick, a SmartMedia and an SD Card.

In accordance with another aspect of the invention, the recording step includes converting raw ECG data into an image file format.

In accordance with another aspect of the invention, an electrotherapy device is provided that includes a controller, an energy source, and at least one electrode for providing electrotherapy to a patient. The device also includes an energy delivery system operable by the controller to deliver an electrical shock from the energy source to the electrode. A processor converts incident data to an image file format and a memory device is provided on which an image file containing the incident data is recorded in the image file format.

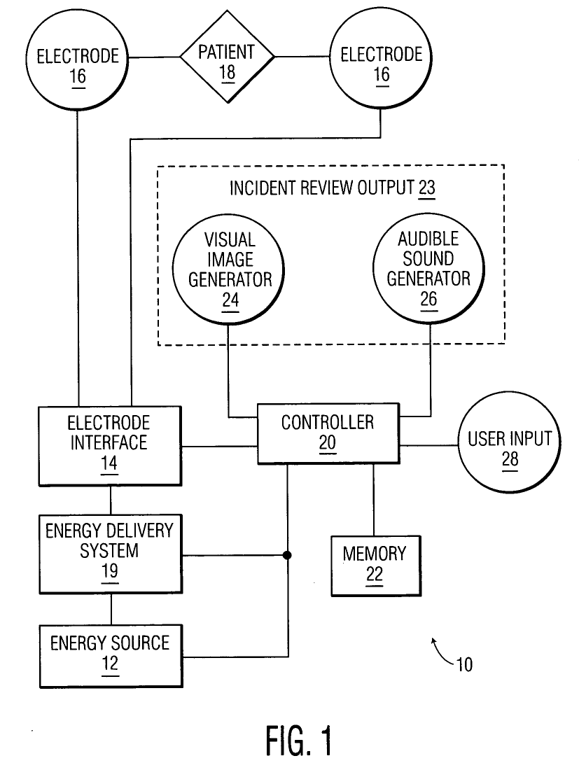

FIG. 1 is a block diagram of a one embodiment of an electrotherapy device constructed in accordance with the present invention.

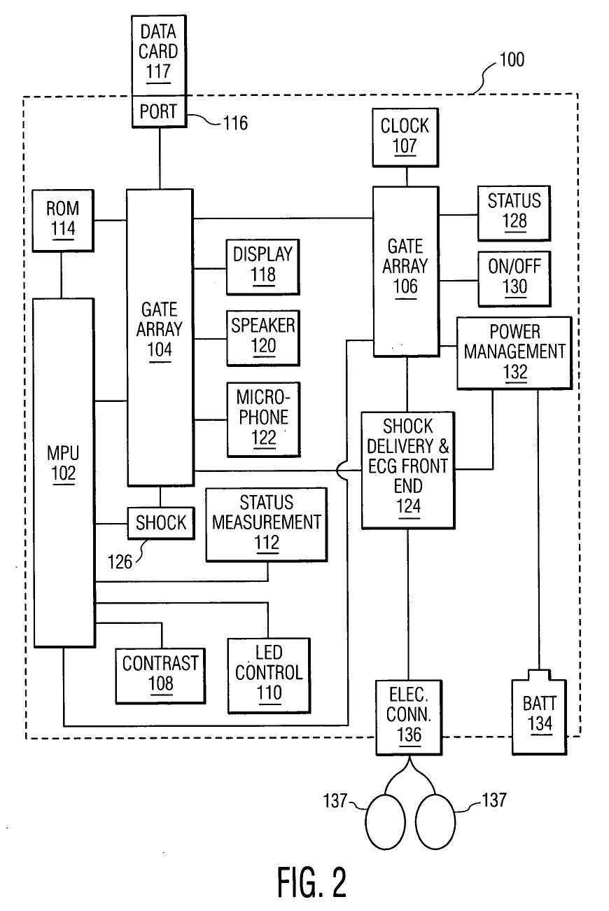

FIGS. 2-4 are a block diagram of alternative embodiments of an electrotherapy device constructed in accordance with the present invention.

The following discussion is presented to enable a person skilled in the art to make and use the invention. Various modifications to the preferred embodiment will be readily apparent to those skilled in the art, and the generic principles defined herein may be applied to other embodiments and applications without departing from the spirit and scope of the present invention as defined by the appended claims. Thus, the present invention is not intended to be limited to the embodiment shown, but is to be accorded the widest scope consistent with the principles and features disclosed herein.

FIG. 1 is a schematic block diagram of a defibrillator system 10 according to a preferred embodiment of this invention. The defibrillator system 10 comprises an energy source 12 to provide voltage or current pulses. A controller 20 operates an energy delivery system 19 to selectively connect and disconnect energy source 12 to and from a pair of electrodes 16 electrically attached to a patient 18 through an electrode interface 14 to provide electrotherapy to the patient. The defibrillator system 10 is an electrotherapy device such as a manual defibrillator or AED. Alternatively, defibrillator system 10 may be a defibrillator trainer that simulates the behavior of a manual or automatic/semi-automatic defibrillator in use, in which case the electrode interface and energy delivery system may be omitted.

In accordance with the present invention, memory 22 is a removable memory card on which data is stored in an image file. Such image files are typically used to store data captured by digital cameras. The data is stored in an image format such as JPEG (Joint Photographic Experts Group). Recently, printers have become available which accept digital camera memory cards in a memory card reader built into the printer. In this way images captured by digital cameras can be printed without the need to first transfer the data from the camera to a computer. Similarly, by incorporating such a removable memory card into an AED and storing the ECG data in an image file, the ECG data can be printed without first being transferred to a computer.

Removable memory card 22 may be any appropriate memory device on which image files can be stored and which is compatible with a memory card reader of the type that is incorporated with a printer. Current examples of such memory devices include, but are not limited to compact FLASH, ATA flash memory cards, Memory Sticks, SmartMedia and SD Cards, which are commercially available from a variety of different vendors.

Of course, to store the ECG data in an image file, the raw data must be converted to an image file. This conversion may be performed by any suitable processor such as a JPEG processing module, for example. The processor may be embodied in hardware, software, firmware, or any combination thereof. The processor may be integrated with the controller 20 or it may be a discrete component. Such processors are well known to those of ordinary skill in the art and thus do not need to be discussed in more detail.

In operation, removable memory card 22 records data collected by the defibrillator while monitoring and treating a patient. Data received with respect to a particular patient's monitoring and treatment is referred to as an “incident.” An incident consists of, for example, discrete events and continuous or non-continuous ECG data. Importantly recorded data may be appended to previously recorded data under appropriate conditions. For example, when a device is deployed to treat a patient, in the event the device were turned off (for example, to replace the battery), the data recorded after the device is turned on would, under some circumstances, be appended to the previously recorded data. Thus, the recorded incident would comprise discrete non-continuous data recorded in connection with a perceived continued use. Alternatively, non-continuous data could result where only a portion of the data received is recorded (for example, data windows around relevant events, e.g. 5 seconds before and after a shock decision).

In some embodiments of the invention the incident also may be reviewed by a subsequent caregiver through an incident review output 23, which consists of a visual image generator 24 and an audible sound generator 26. Visual image generator 24 and audible sound generator 26 are discussed in more detail in the aforementioned U.S. Published Patent Application No. 2002/0156503. Visual image generator 24 may display, among other things, current ECG, ECG history, etc. The visual image generator 24 may be, for example, a liquid crystal display (“LCD”). Activation of the visual image generator 24 and the audible sound generator 26 is controlled by the controller 20 in response to the information received from memory 22. A user input 28 may also provided to interact with the memory 22 to control the incident review. Visual image generator 24, audible sound generator 26, and user input 28 are discussed in more detail in the aforementioned U.S. Published Patent Application No. 2002/0156503.

The major components of a semi-automatic external defibrillator according to another embodiment of the invention are shown in FIG. 2 in block diagram form. Defibrillator control functions are divided among a microprocessor unit (MPU) 102 and two custom gate arrays 104 and 106. It should be understood, however, that gate arrays 104 and 106 are optional, and their functions can be performed by other circuits.

MPU 102 performs program steps according to software instructions provided to it from ROM 114. MPU 102 controls the operation of certain buttons (such as display contrast buttons 108) and certain system LED's 110 (such as LED's associated with the shock button and the electrode connector). MPU 102 also receives system status information as shown by block 112. MPU 102 also controls the operation of the display contrast button 108 while functioning as soft keys.

Gate array 104 implements the memory map to system ROM 114, data card port 116 and other system memory elements. System ROM 114 is preferably flash ROM, although EPROM or any other electrically erasable and programmable nonvolatile memory could be used. As in the embodiment of the invention shown in FIG. 1, a data card port 116 is provided in which a removable memory card 117 can be located. The removable memory card 117 stores the ECG data in image files so that the data can be directly printed out when the removable memory card is inserted into the memory card reader of a printer. As previously mentioned, current examples of such removable memory cards include compact FLASH, ATA flash memory cards, Memory Sticks, SmartMedia and SD Cards.

For purposes of writing to the removable memory card, gate array 104 provides the interface and control between defibrillator 100 and the data card 117 attached to the data card port 116. For example, gate array 104 contains a FIFO buffer to compensate for differences between the speed with which ROM 114 can be accessed by MPU 102 and the speed with which the memory portion of data card 117 can be accessed. Gate array 104 may also serve as the processor for converting the raw ECG data to an image file. Alternatively, the ECG data may be converted to an image file by any other suitable component. Gate array 104 also controls a display 118, a speaker 120, and a microphone 122. Gate array 104 can actuate a relay within the shock delivery and ECG front end system 124 in response to actuation of a shock button 126 by a user during treatment mode.

Gate array 106 receives time information from clock 107. Gate array 106 also provides a system monitor function by performing automatic self-tests of the defibrillator and its components. The gate array 106 displays the operational status of the defibrillator on a status display 128. Gate array 106 is also the defibrillator's interface with a user-activated on/off switch 130.

Gate array 106 controls the power management subsystem 132 to provide power to operate system components from battery 134 and to provide energy to the shock delivery system's capacitor(s) for a therapeutic shock during treatment mode. Gate array 106 also interfaces with the defibrillator's ECG front end 124, enables the shock delivery system to deliver a shock in response to detection of a patient ECG pattern requiring treatment (and actuation of the shock button), and controls delivery of the shock to electrode connector 136 in response to shock delivery status information obtained during delivery of the shock. Further information regarding this last function may be found in U.S. Pat. No. 5,735,879 to Gliner et al. for “Electrotherapy Method for External Defibrillators” and U.S. Pat. No. 5,607,454 to Cameron et al. for “Electrotherapy Method and Apparatus,” the disclosures of which are incorporated herein by reference.

As is known in the art, the external defibrillator 100 can be operated in different modes, such as self-test mode, stand-by mode and patient treatment and monitoring mode. Further discussion of the operation of an external defibrillator in self-test mode, stand by mode and patient treatment mode is provided in, for example, U.S. Pat. No. 5,800,460, to Powers et al. for “Method for Performing Self-Test In A Defibrillator,” the specification of which is incorporated herein.

During patient treatment and monitoring mode, the defibrillator receives ECG information from a patient through electrodes 137. The defibrillator then analyzes the ECG information to determine whether a therapeutic shock is advised. In a semi-automatic external defibrillator, a shock is delivered to the patient through the electrodes if a shock is advised and if the shock button 126 is actuated by a user. In a fully automatic external defibrillator, a shock would be delivered to the patient without further user intervention.

This information and sequence of events is stored by the defibrillator in removable memory card 117 as an image file. In addition to recording patient ECG information, and defibrillator operation information, the defibrillator may also record other information (such as ambient sounds received by microphone 122).

The incident data collected by an external defibrillator (such as the defibrillator of FIG. 2) in patient treatment mode according to a preferred embodiment of this invention includes the following: defibrillator power on; defibrillation pads on or off; patient ECG; artifact detection; shock advised; no shock advised; charge begun; charge complete; device armed; device disarmed; shock initiated; shock delivered; shock aborted; pause for CPR; pause ended; manual override; manual charge; manual timeout; device off; low battery; depleted battery; critical error detected; non-critical error detected; audio (e.g., voice). The defibrillator may also obtain time information from, for example, the defibrillator clock, and/or the clock on the memory device, and store the time information with the incident data.

### Other Embodiments

Printers and other devices, such as personal digital assistants and cellular telephones, have become available which directly receive image data via wireless means and display those images without requiring an intermediate computer. Accordingly, in some embodiments of the invention image data files could thus be communicated to such a display device directly via wireless means such as infrared or radio frequency (RF) communications.

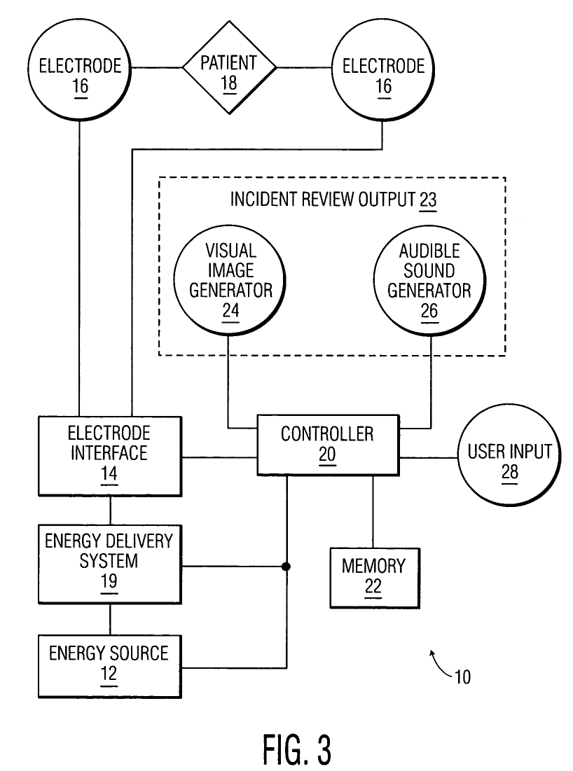

FIG. 3 is a schematic block diagram of a defibrillator system 10′ according to an alternative embodiment of the invention. Defibrillator system 10′ is disposed like and functions similarly to defibrillator system 10, except that fixed memory 22′ stores data in an image file. Such image files are typically used to store data captured by digital cameras. The data is stored in an image format such as JPEG (Joint Photographic Experts Group). Instead of a removable memory device, the image file is transmitted via wireless means to the external printing or display device.

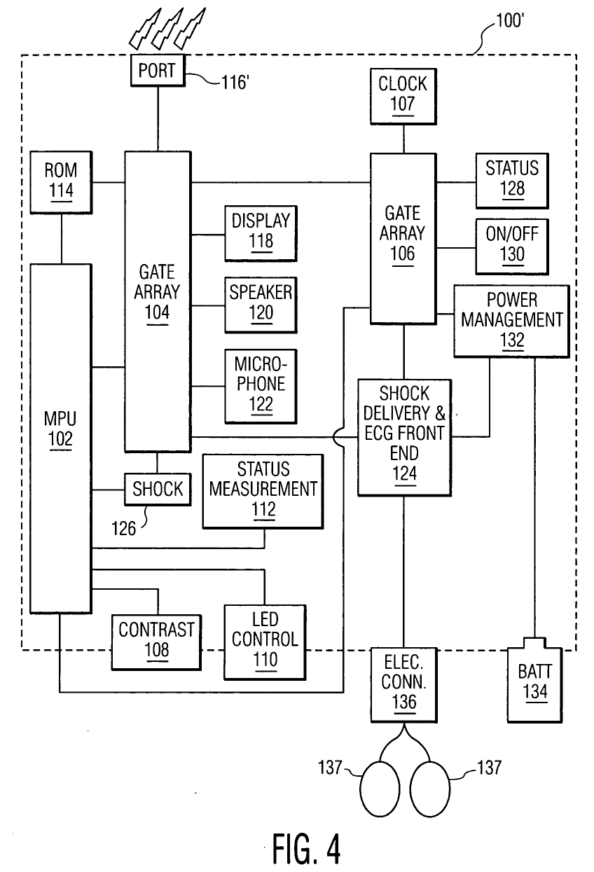

The major components of a semi-automatic external defibrillator 100′ according to the alternative embodiment of the invention depicted in FIG. 3 are shown in FIG. 4 in block diagram form. Defibrillator 100′ is disposed like and functions similarly to defibrillator 100 of FIG. 2, except that a port 116′ transmits the image file to the external printing device or display device. Port 116′ transmission means is preferably by infrared serial communications, but can also be a radio frequency transmission, such as Bluetooth wireless transmission.

By transmitting image files directly from the defibrillator 100′ to a display device, a computer is not required. It is further possible in this way to display image files on display devices which are at the scene of the rescue, thus providing on-scene rescuers with important ECG information.

1. A method of reviewing incident data on an external defibrillator (100) comprising:
deploying the defibrillator (100) for use in an emergency, wherein the defibrillator (100) is attached to a patient (18);
monitoring ECG data from the patient; and
recording the monitored ECG data in an image file located in a memory device (22) incorporated in the defibrillator (100).

2. The method of claim 1 wherein said memory device (22) is user removable, and further comprising the steps of printing the recorded ECG data by removing the memory device (22) from the defibrillator (100) and inserting the memory (22) into a memory reader incorporated in a printer.

3. The method of claim 1 wherein the recording step includes recording audible data received from a microphone (122).

4. The method of claim 1 wherein the recording step includes recording defibrillator operation information.

5. The method of claim 1 wherein said external defibrillator (100) is a fully automatic external defibrillator.

6. The method of claim 1 wherein said external defibrillator (100) is a semi-automatic external defibrillator.

7. The method of claim 1, further comprising the step of transmitting said recorded ECG image file from the defibrillator (100) to a display device (118).

8. The method of claim 1 further comprising the step of selecting additional data to be recorded in the image file located on the memory (22).

9. The method of claim 8 wherein said additional data is selected from the group consisting of audible data and patient therapy data.

10. The method of claim 2 wherein said memory device (22) is selected from the group consisting of a compact FLASH, an ATA flash memory card, a Memory Stick, a SmartMedia and an SD Card.

11. The method of claim 1 wherein the recording step includes converting raw ECG data into an image file format.

12. An electrotherapy device comprising:
a controller (20);
an energy source (12);
at least one electrode (16) for providing electrotherapy to a patient (18);
an energy delivery system (19) operable by the controller (20) to deliver an electrical shock from the energy source (12) to the at least one electrode (16);
a processor for converting incident data to an image file format; and
a memory device (22) on which an image file containing the incident data is recorded in the image file format.

13. The electrotherapy device of claim 12 wherein the incident data includes ECG data.

14. The electrotherapy device of claim 12 wherein said memory device (22) is user removable and is adapted to be read by a memory reader incorporated in a printer.

15. The electrotherapy device of claim 12 wherein the incident data includes audible data received from a microphone (122).

16. The electrotherapy device of claim 12 wherein the incident data includes defibrillator operation information.

17. The electrotherapy device of claim 12 wherein said electrotherapy device is an external defibrillator (100).

18. The electrotherapy device of claim 17 wherein said external defibrillator (100) is a fully automatic external defibrillator.

19. The electrotherapy device of claim 17 wherein said external defibrillator (100) is a semi-automatic external defibrillator.

20. The electrotherapy device of claim 12 further comprising a port (116) disposed to transmit said image file.

21. The electrotherapy device of claim 12 wherein said incident data is selected from the group consisting of ECG data, audible data and patient therapy data.

22. The electrotherapy device of claim 14 wherein said user removable memory device (22) is selected from the group consisting of a compact FLASH, an ATA flash memory card, a Memory Stick, a SmartMedia and an SD Card.
